(19) United States
(12) Patent Application Publication
(10) Pub. No.: US 2005/0228388 A1
(43) Pub. Date: Oct. 13, 2005

(54) DOUBLE LEAD BONE SCREW

(76) Inventors: Darrel Brodke, Salt Lake City, UT (US); G. Kris Kumar, Raynham, MA (US); Michael Varieur, Portsmouth, RI (US)

Correspondence Address: NUTTER MCCLENNEN & FISH LLP, WORLD TRADE CENTER WEST, 155 SEAPORT BOULEVARD, BOSTON, MA, 02210-2604, US

(21) Appl. No.: 10/708,881

(22) Filed: Mar. 30, 2004

(51) Int. Cl. A61B 17/58

(52) U.S. Cl. 606/73

(57) ABSTRACT

A bone screw is provided having a head and a shank with opposed first and second helical threads extending there around. In one embodiment, the shank has a proximal portion with a constant minor diameter, and a distal portion with a minor diameter that decreases in a proximal-to-distal direction to form a taper. The opposed first and second helical threads preferably define a thread depth that remains constant along the length of the shank. In an exemplary embodiment, a major diameter of the shank at a distal tip of the shank is equal to or less than the minor diameter of the proximal portion of the shank.

### FIELD OF THE INVENTION

The present invention relates to bone screws, and in particular, to a bone screw having improved physical and mechanical properties.

### BACKGROUND OF THE INVENTION

Bone screws are used for a variety of medical purposes, including to correct spinal pathologies, deformities, and trauma. Spinal bone screws are loaded with axial, distractive, and compressive forces, and with subsequent cyclically loaded forces applied through the patient's natural movement. Thus, spinal bone screws must be sufficiently strong, while at the same time they must be designed to minimize potential damage to the bone.

Conventional bone screws are typically made from a cylindrical or tapered core having a helical thread with either a variable or a constant major diameter extending along the entire length of the screw. The helical shape of the threads cuts a path into the bone as the screw rotates, and prevents the screw from being axially pulled out of the bone. Thus, threads having relatively deep flanks and/or a small core diameter will increase the pull-out strength of the screw. Conventional bone screws, however, typically require a relatively large core diameter to withstand high torque without shearing or otherwise failing. A thick core can, however, displace enough bone to cause the bone to split or otherwise become damaged. One other drawback of conventional bone screws is that the single helical thread results in a slower insertion rate, which can be dissatisfying to many surgeons.

Accordingly, there is a need for an improved bone screw having a high pull-out strength, that is easy to implant, that provides a reduced insertion time, and that facilitates insertion at an optimum trajectory.

### BRIEF SUMMARY OF THE INVENTION

The present invention provides a bone screw that is particularly useful as a spinal screw. In general, the bone screw has a dual-lead shank with a tapered distal portion. The distal portion allows the screw to be self-introduced into bone, and it is also adapted to guide the screw towards an optimum trajectory. In one embodiment, the bone screw includes a head, and a shank having a proximal portion with a constant minor diameter, and a distal portion with a minor diameter that decreases in a proximal-to-distal direction. In an exemplary embodiment, the minor diameter at the proximal portion of the shank is in the range of about 3 mm to 5 mm, and the minor diameter at the distal portion of the shank is less than the minor diameter at the proximal portion of the shank. The bone screw also includes opposed first and second helical threads that extend around the length of the shank and that define a thread depth that remains constant along the length of the shank. In an exemplary embodiment, a major diameter of the shank at a distal tip of the shank is equal to or less than the minor diameter of the proximal portion of the shank.

While the bone screw can have a variety of shapes and sizes, in a preferred embodiment the distal portion of the shank has a length that is at least about 10% of the length of the shank, but more preferably the length of the distal portion is about 10 mm. In an exemplary embodiment, the length of the shank is in the range of about 20 mm to 100 mm.

In another embodiment of the present invention, a root of each of the opposed first and second helical threads can have a width extending between proximal and distal facing flanks that remains substantially constant along the length of the shank. A crest of each of the opposed first and second helical threads can also have a width extending between proximal and distal facing flanks that remains substantially constant along the length of the shank. In an exemplary embodiment, the width of the crest is about 0.2 mm. The bone screw also preferably has a pitch that is about 6 mm.

In yet another embodiment of the present invention, a bone screw is provided having a head with a driver-receiving element formed thereon, and a shank formed from first and second axially symmetrical threads offset approximately 180° from one another and extending around the shank between proximal and distal ends thereof. The threads preferably have a depth that remains substantially constant along a length of the shank. A proximal portion of the shank can have a minor diameter that is equal to or greater than a major diameter of the shank at a distal-most end thereof. In an exemplary embodiment, a proximal portion of the shank has a constant minor diameter, and a distal portion of the shank has a minor diameter that decreases in a proximal-to-distal direction.

### BRIEF DESCRIPTION OF THE DRAWINGS

The invention will be more fully understood from the following detailed description taken in conjunction with the accompanying drawings, in which:

FIG. 1 is a perspective view of a bone screw according to one embodiment of the present invention having a proximal portion with a constant minor diameter, and a distal portion with a tapered minor diameter;

FIG. 2A is a side view of the bone screw shown in FIG. 1;

FIG. 2B is a cross-sectional view of one of the threads of the bone screw shown in FIG. 2A; and

FIG. 3 is a cross-sectional view of the bone screw shown in FIG. 1.

### DETAILED DESCRIPTION OF THE INVENTION

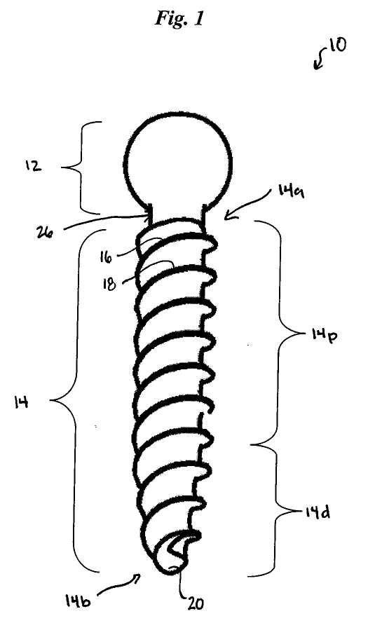

In general, as shown in FIGS. 1-3, the present invention provides a bone screw 10 having a head 12 that can be adapted to mate with a driver tool, and a shank 14 having proximal and distal ends 14a, 14b. First and second helical threads 16, 18 extend around the shank 14 between the proximal and distal ends 14a, 14b thereof, and the threads 16, 18 are axially symmetrical and offset approximately 180° from one another. The shank 14 also includes proximal and distal portions 14p, 14d that differ from one another, and that are particularly adapted to facilitate use of the bone screw 14 in a patient's spinal column. In particular, the proximal and distal portions 14p, 14d are configured to facilitate relatively quick and easy insertion of the bone screw 10 into bone, and to provide adequate fixation once implanted.

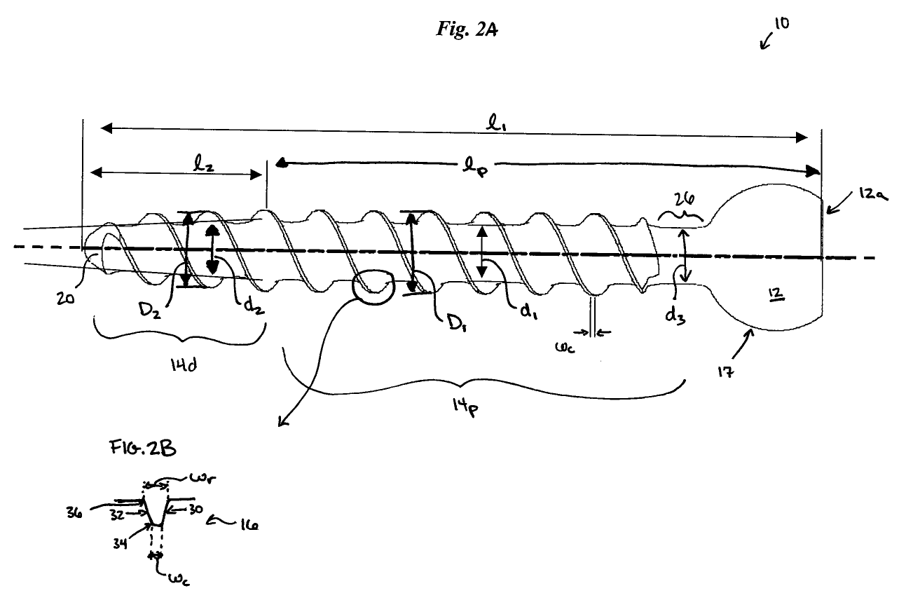

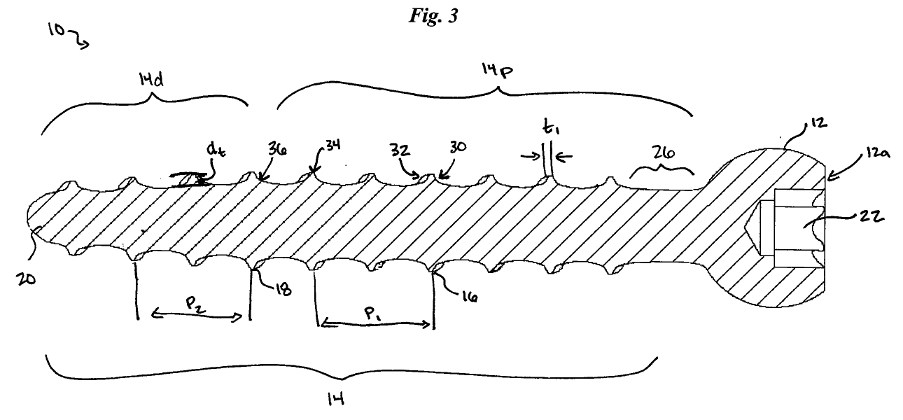

The head 12 of the bone screw 10 can have a variety of configurations, and it can be adapted for a variety of uses. As shown in FIGS. 2A-3, the head 12 of the bone screw 10 has a substantially spherical mating surface 17, but it includes a flattened proximal surface 12a. A driver-receiving element 22 (shown in FIG. 3) is formed in the proximal surface 12a of the head 12 and it is adapted to mate to a driver tool for driving the bone screw 10 into bone. The driver-receiving element 22 can have a variety of configurations, and FIG. 3 merely illustrates one embodiment of a driver-receiving element 22 that is in the form of a six-pointed star-shaped socket for receiving a complementary-shaped driver member. A person skilled in the art will appreciate that a variety of driver-receiving elements can be used, and that the head 12 of the bone screw 10 can have virtually any configuration.

As previously stated above, the shank 14 of the bone screw 10 includes proximal and distal portions 14p, 14d that differ with respect to one another. Referring to FIG. 2A, While the length of the proximal and distal portions 14p, 14d can vary depending on the size of the screw 10 and the intended use, in an exemplary embodiment the distal portion 14d preferably has a length l₂ that is at least about 10% of the entire length l of the bone screw 10. More preferably, however, the length l of the distal portion 14d is about 10 mm, regardless of the length l of the bone screw 10, which preferably ranges from about 20 mm to 100 mm. As is further shown in FIG. 2A, the proximal portion 14p of the bone screw 10 can have a minor diameter d that preferably remains substantially constant along a length l thereof, while the distal portion 14d has a minor diameter d that decreases in a proximal-to-distal direction to form a taper. The taper facilitates insertion of the distal portion 14d into bone, and it can also be effective to guide the bone screw 10, preventing misalignment and guiding the bone screw toward an optimal trajectory.

The opposed helical threads 16, 18 that extend around and along the shank 14 each preferably begin at the head 12 of the screw 10, or at a position just distal to the head 12, and they terminate at an apex 20 that forms distal tip of the screw 10. The threads 16, 18 can also terminate at a position just proximal to the apex 20 of the screw 10 depending on the configuration of the apex 20, which will be discussed in more detail below. In an exemplary embodiment, as shown in FIGS. 1-3, the helical threads 16, 18 start at a position spaced apart from the head 12 such that the bone screw 10 includes a thread-free shank portion 26. Since the illustrated bone screw 10 is a polyaxial screw, the thread-free shank portion 26 allows the screw 10 to rotate within a screw-receiving bore formed in another medical implant, such as a rod-receiving head of a spinal anchor. The thread-free portion 26 of the shank 14 can have any diameter d₃, but preferably the diameter d₃ of the thread-free portion 26 is the same as or less than the minor diameter d₁ of the proximal portion 14p of the shank 14.

As noted above, the helical threads 16, 18 preferably start at a position approximately 180° apart from one another on the shaft and terminate at or adjacent to an apex 20 that forms the distal tip of the screw 10. The apex 20 can have a variety of configurations. By way of non-limiting example, the apex 20 can be in the form of a cone-type or gimlet-type tip. As shown in FIGS. 1-3, the apex 20 of the screw 10 is in the form of a gimlet tip, wherein the threads 16, 18 extend to and merge at the distal tip of the screw 10. As a result, the bone screw 10 is a self-tapping screw, which in many cases may eliminate the use of a tap prior to threading the screw 10 into the bone. With cone-type tips, the threads 16, 18 terminate at a position just proximal to the distal tip of the screw, and the tip 20 is formed into a solid, cone-like structure. A person skilled in the art will appreciate that either tip can be used, or alternatively the apex 20 can have a variety of other configurations.

The threads 16, 18 of the bone screw 10 can also have a pitch P that varies depending upon the requirements of a given screw. Referring to FIG. 3, the pitch P is determined by the distance between the threads 16, 18 on one helix, thus the bone screw 10 can have a first pitch P for the first thread 16 and a second pitch P for the second thread 18. In an exemplary embodiment, the pitch P, P for each thread 16, 18 is the same and is in the range of about 4 mm to 8 mm, and more preferably is about 6 mm.

As is further shown in FIGS. 1-3, each thread 16, 18 includes a proximal facing flank 30, a distal facing flank 32, a crest 34, and a root 36. Since the threads 16, 18 are substantially identical to one another, only single reference numbers will be used to describe features of each of the threads 16, 18. Referring to FIG. 3, the proximal and distal facing flanks 30, 32 of the threads 16, 18 define a thickness t₁ which can vary along the length l₁ of the bone screw 10, as well as between the root 36 and the crest 34 of each thread 16, 18. In an exemplary embodiment, however, the thickness t₁ of the threads 16, 18 remains substantially constant along the length l₁ of the bone screw 10, and it preferably only varies between the root 36 and the crest 34 of the threads 16, 18, decreasing gradually from root 36 to crest 34. This can be achieved by forming proximal and distal facing flanks 30, 32 that converge toward one another between the root 34 and the crest 36 of the threads 16, 18 such that the crest 36 has a width w that is less than a width w of the root 34, as shown in FIG. 2B, which illustrates a cross-section of one of the threads, e.g., thread 16. While the angle of convergence can vary between the proximal and distal facing flanks 30, 32, in an exemplary embodiment the flanks 30, 32 converge toward one another at the same angle. In another embodiment, the thickness t of the threads 16, 18 can vary depending on the size of the bone screw 10, but the thickness t is preferably less than the smallest minor diameter, e.g., the minor diameter d at the distal end 14b of the shank 14, and more preferably the thickness t of the threads 16, 18 is in the range of about 0.15 to 0.30 mm, and more preferably is about 0.2 m.

While a major portion of the proximal and distal facing flanks 30, 32 preferably converge toward one another, the threads 16, 18 can, however, include a crest 34 formed from an outer-most portion of the proximal and distal facing flanks 30, 32 that varies in shape and size. For example, the crest 34 can form a sharp edge or a beveled edge. In an exemplary embodiment, as shown in FIG. 2B, the proximal and distal facing flanks 30, 32 terminate at a crest 34 that is substantially flat such that the crest 34 is substantially parallel to the root 36 or shank 14 of the bone screw 10. The width w_{c} of the crest 34, which is measured by the distance between the proximal and distal facing flanks 30, 32, preferably remains substantially constant along the length of the shank 14. While not illustrated, the crest 34 can have a variety of other configurations, and the crest 34 and root 36 can be positioned at various angles relative to one another. Moreover, the crest 34 can have a width w_{c} that is substantially the same as the thread thickness t.

The bone screw 10 also includes a major diameter which is defined by the distance between opposed crests 34 of the threads 16, 18. The major diameter of the bone screw 10 preferably varies between the proximal and distal portions 14p, 14d of the bone screw 10. In an exemplary embodiment, as shown in FIG. 2A, the proximal portion 14p has a major diameter D₁ that remains substantially constant along a length of the proximal portion 14p of the screw, and the distal portion 14d has a major diameter D that decreases in a proximal-to-distal direction. The rate of decrease, e.g., the taper rate, of the major diameter D of the distal portion 14d is preferably the same as the taper rate of the minor diameter d of the distal portion 14d. As a result, the threads 16, 18 have a depth d (FIG. 3) that is constant along the entire length l of the bone screw 10. In an exemplary embodiment, the distal portion 14d tapers at a rate that results in the distal portion 14d having a major diameter D that is less than or equal to a minor diameter d of the proximal portion 14p of the bone screw 10. Such a configuration is particularly advantageous because, when the bone screw 10 is implanted in bone, the hole created by the distal portion 14d of the shank 14 will have a diameter than is less than or equal to a minor diameter d of the proximal portion 14p of the bone screw 10 to facilitate insertion of the screw 10. In an exemplary embodiment, the taper rate is in the range of about 0.5° to 15°.

In use, the bone screw 10 is driven into bone, such as vertebral bone, using a driver tool that mates with the hexagonal socket 22 in the head 12 of the screw 10. As the screw 10 is inserted into the bone, the threads 16, 18 will cut through the bone in a helical pattern such that an area between the threads 16, 18 will be filled with bone. This will prevent the screw 10 from being pulled out of the bone, and will reduce the amount of damage to the bone surrounding the screw 10, as less bone needs to be displaced to implant the screw 10. When used in vertebral bone, the distal portion 14d of the bone screw 10 will extend into the vertebral body, while the remainder of the bone screw 10 will be disposed in pedicle bone. This is particularly desirable, as the strongest part of the screw 10, which is the proximal portion 14p of the screw 10, needs to be in pedicle bone.

The bone screw according to the present invention can be made from any biocompatible material, including biocompatible metals and polymers. It is also contemplated that the bone screw can equally comprise bioabsorbable and/or biodegradable materials. Suitable materials include, but are not limited to, all surgically appropriate metals including titanium, titanium alloy, chrome alloys and stainless steel, and non-resorbable non-metallic materials such as carbon fiber materials, resins, plastics and ceramics. Exemplary materials include, but are not limited to, PEAK, PEEK, PEK, PEKK and PEKEKK materials net or reinforced with, for example, carbon fibers or glass fibers. A person skilled in the art will appreciate that any number of a wide variety of materials possessing the mechanical properties suitable for attachment with bone can be used.

One of ordinary skill in the art will appreciate further features and advantages of the invention based on the above-described embodiments. Accordingly, the invention is not to be limited by what has been particularly shown and described, except as indicated by the appended claims. All publications and references cited herein are expressly incorporated herein by reference in their entirety.

1. A bone screw, comprising:
a head;
a shank having a proximal portion with a constant minor diameter, and a distal portion with a minor diameter that decreases in a proximal-to-distal direction; and
opposed first and second helical threads, the threads extending around at least a portion of a length of the shank and defining a thread depth that remains constant along the length of the shank.

2. The bone screw of claim 1, wherein a major diameter of the shank at a distal tip of the shank is equal to or less than the minor diameter of the proximal portion of the shank.

3. The bone screw of claim 1, wherein the distal portion of the shank has a length that is about 10 mm.

4. The bone screw of claim 1, wherein the distal portion of the shank has a length that comprises at least about 10% of a length of the bone screw.

5. The bone screw of claim 1, wherein the bone screw has a length in the range of about 20 mm to 100 mm, and the distal portion of the shank has a length of about 10 mm.

6. The bone screw of claim 1, wherein a root of each of the opposed first and second helical threads has a width extending between proximal and distal facing flanks that remains substantially constant along the length of the shank.

7. The bone screw of claim 1, wherein a crest of each of the opposed first and second helical threads has a width extending between proximal and distal facing flanks that remains substantially constant along the length of the shank.

8. The bone screw of claim 7, wherein the width of the crest is about 0.2 mm.

9. The bone screw of claim 1, wherein the opposed first and second helical threads define a pitch of about 6 mm.

10. The bone screw of claim 1, wherein the opposed first and second helical threads each have proximal and distal flanks that converge toward one another from a root to a crest thereof.

11. The bone screw of claim 10, wherein the proximal and distal flanks converge toward one another at substantially the same rate.

12. The bone screw of claim 1, wherein the opposed first and second helical threads each have proximal and distal flanks that converge toward one another at an outer-most crest thereof to form a flat edge.

13. The bone screw of claim 1, wherein the minor diameter at the proximal portion of the shank is in the range of about 3 mm to 5 mm, and wherein the minor diameter at the distal portion of the shank is less than the minor diameter at the proximal portion of the shank.

14. The bone screw of claim 1, further comprising a distal tip formed on a distal-most end of the shank.

15. The bone screw of claim 14, wherein the distal tip is a self-tapping tip.

16. A bone screw, comprising:
a head having a driver-receiving element formed thereon;
a shank formed from first and second axially symmetrical threads offset approximately 180° from one another and extending around at least a portion of the shank between proximal and distal ends thereof, the threads having a depth that remains constant along a length of the shank, and a proximal portion of the shank having a minor diameter that is equal to or greater than a major diameter of the shank at a distal-most end thereof.

17. The bone screw of claim 16, wherein a proximal portion of the shank has a substantially constant minor diameter, and a distal portion of the shank has a minor diameter that decreases in a proximal-to-distal direction.

18. The bone screw of claim 16, wherein the distal portion of the shank has a length that is at least about 10% of a length of the bone screw.

19. The bone screw of claim 16, wherein the distal portion of the shank has a length that is about 10 mm.

20. The bone screw of claim 16, wherein the bone screw has a length in the range of about 20 mm to 100 mm, and the distal portion of the shank has a length of about 10 mm.

21. The bone screw of claim 16, wherein a root of the threads has a width extending between proximal and distal facing flanks that remains substantially constant along the length of the shank.

22. The bone screw of claim 16, wherein a crest of each the threads has a width extending between proximal and distal facing flanks that remains substantially constant along the length of the shank.

23. The bone screw of claim 22, wherein the width of the crest is about 0.2 mm.

24. The bone screw of claim 16, wherein the threads define a pitch of about 6 mm.

25. The bone screw of claim 16, wherein the threads each have proximal and distal flanks that converge toward one another from a root to a crest thereof.

26. The bone screw of claim 25, wherein the proximal and distal flanks converge toward one at substantially the same rate.

27. The bone screw of claim 16, wherein the threads each have proximal and distal flanks that converge toward one another at an outer-most crest thereof to form a flat edge.

28. The bone screw of claim 16, further comprising a distal tip formed on a distal-most end of the shank.

29. The bone screw of claim 28, wherein the distal tip is a self-tapping tip.

30. A bone screw, comprising:
a head;
a shank having a proximal portion with a constant minor diameter, and a distal portion with a minor diameter that decreases in a proximal-to-distal direction; and
opposed first and second helical threads formed on at least a portion of the shank and defining a major diameter that decreases at the same rate as the minor diameter of the shank.
